(19) United States
(12) Patent Application Publication
(10) Pub. No.: US 2004/0138703 A1
(43) Pub. Date: Jul. 15, 2004

(54) Seal for posterior lateral vertebral disk cavity

(76) Inventor: Neville Alleyne, La Jolla, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/655,852

(22) Filed: Sep. 5, 2003

Related U.S. Application Data
(60) Provisional application No. 60/409,125, filed on Sep. 6, 2002.

(51) Int. Cl. A61F 2/44

(52) U.S. Cl. 606/213; 623/17.16; 606/151

(57) ABSTRACT

A biocompatible protection device and method of utilizing the device in conjunction with bone morphogenic protein is disclosed to inhibit the postoperative formation bone outside of an intended location. The device comprises a seal contoured to a posterior lateral vertebral cavity to inhibit migration of the bone morphogenic protein from the cavity.

### RELATED APPLICATIONS

This application claims priority to U.S. Provisional Application No. 60/409,125, filed Sep. 6, 2002, which is incorporated by reference in its entirety.

### BACKGROUND OF THE INVENTION

1. Field of the Invention

This invention relates generally to orthopedic surgery devices, and more specifically to a surgical device suitable for retaining a bone morphogenic protein within a desired location. It also relates to methods for inhibiting leakage or migration of bone morphogenic protein outside of an introduction site and to methods for preventing bone formation outside of the site of bone morphogenic protein introduction.

2. Description of the Related Art

Intervertebral discs, located between the endplates of adjacent vertebrae, stabilize the spine and distribute forces between vertebrae and cushion vertebral bodies. A normal intervertebral disk is comprised a semi-gelatinous component, the nucleus pulposus, which is surrounded and confined by an outer, fibrous ring called the annulus fibrosis. The annulus portion is comprised of collagen fibers that may weaken, rupture, or tear. This degeneration can produce disk bulges, herniations, and other disk pathologies. The bulge or herniation may press on a spinal nerve causing nerve irritation or damage with resultant back pain and/or weakness and pain in the extremities.

Typical spinal fusion procedures entail surgical removal of a portion or all of an intervertebral disk followed by fusion of the adjacent vertebrae. Following this removal, a bone fusing medium is introduced between the adjacent vertebrae to prevent collapse and promote fusion of the adjacent vertebrae.

Bone grafts are commonly accomplished by using bone material harvested from elsewhere on the patient's body. However, the harvesting procedure may cause the patient as much, or more, pain than the fusion surgery. More recently, Bone morphogenic proteins (BMPs), a class of osteoinductive factors from bone matrix, which are capable of inducing bone formation when introduced into a surgical site have been developed.

BMPs, however, also have a disadvantage in that BMP may migrate from the area into which it has been introduced and cause unintended bone growth elsewhere. When BMP is used, for example, in a vertebral fusion procedure in place of a bone graft, the BMP may be introduced into the disk space. Once introduced, the BMP induces ectopic bone formation. Bone is thereby formed between the adjacent vertebrae, fusing them together.

If, however, BMP leaks from the annulus, bone may form in other than the intended area. This unintended bone growth may exert pressure on nearby nerves, thereby causing more pain for the patient.

A need therefore exists for a device and method for inhibiting the migration of BMP from the intended target site and thus avoid this potential disadvantage of BMP use.

### SUMMARY OF THE INVENTION

A device in accordance with the present invention comprises a seal formed of a solid sheet, the sheet having a protruding portion configured to fit in an opening in a disk annulus.

This invention also relates to a method of using BMP. A posterior lateral herniation in a spinal disk annulus is resected to form an opening in a posterial lateral location of the disk annulus. BMP is introduced into this opening. The opening is sealed with a biocompatible seal which is positioned to cover and seal the opening so as to inhibit leakage of BMP into surrounding material.

### BRIEF DESCRIPTION OF THE DRAWINGS

FIG. 1 depicts a top cross-sectional view of a spinal disk following removal of a herniation.

FIG. 2 depicts a top cross-sectional view of a spinal disk following positioning of a seal according to the present invention.

FIG. 3A illustrates a top perspective view of one embodiment of a seal device.

FIG. 3B depicts a side cross-sectional view of one embodiment of a seal device.

### DETAILED DESCRIPTION OF THE PREFERRED EMBODIMENT

A number of back surgery procedures, laminectomies, hemilaminectomies spinal stenosis surgery, and diskectomies, including microdiskectomies, involve the removal of vertebral bone and disk tissue. Following such a bony dissection, a bone fusing medium may be introduced between the adjacent vertebrae in order to fuse the adjacent vertebrae together. Where BMP is introduced as the bone forming medium, the present invention provides a seal to inhibit migration of the BMP into surrounding tissues where unintended bone formation may press on a nearby nerve and cause pain.

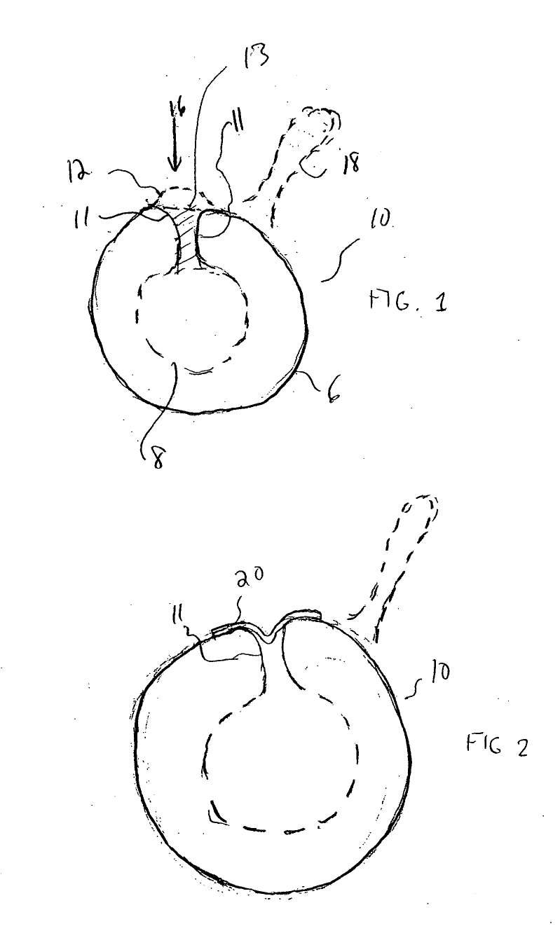

In accordance with one embodiment of a method of the present invention, FIG. 1 depicts a top cross-sectional view of a spinal disk 10. The disk is comprised of an outer, fibrous ring, the annulus fibrosis 6 and an inner semi-gelatinous component, the nucleus pulposus 8. The spinal process 18 is a portion of the vertebrae that protrudes posteriorly from the spinal column. A herniation 12 is depicted in a posterior lateral position on the annulus 6.

According to an embodiment of the present invention, the herniation 12 on the posterior lateral region of a disk is resected. A new surface is thereby exposed on the annulus 6. The new surface forms the wall 11 of an opening or cavity 13 in a posterior lateral location of the annulus. BMP is introduced into this posterior lateral cavity through means such as injection from a syringe into the cavity 13 in the direction of arrow 16.

Positioning a seal 20 as depicted in FIG. 2 then seals the cavity 13. When positioned, the outer portion of the seal 20 may be positioned in contact with the outer surface of the spinal disk 10 such that the contours of the seal are substantially aligned to the contours of the surface of the annulus 6 in proximity to the cavity 13. A protruding portion of surface of the seal device 20 may be aligned with a portion of the wall 11 and be received into the cavity 13. The device 20 thereby forms a seal that inhibits the migration of the BMP into the surrounding tissue where unintended bone formation may otherwise result.

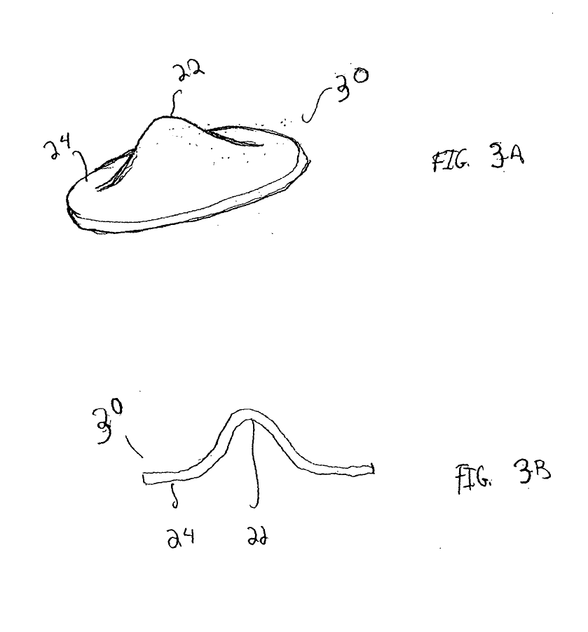

As noted, the seal surface is contoured to align with the surface of the disk and vertebral bodies, in and around, the disk cavity 13. FIG. 3A illustrates a top perspective view of a seal device according to one embodiment of the present invention. The seal is comprised of a sheet 30. An edge portion 24 the sheet 30 may be contoured to align with the external surface proximal vertebral disk in proximity to the cavity 13 formed in the annulus 6. A central portion 22 of the sheet 30 may be contoured to follow the cavity wall 11 and thereby seal the BMP in the cavity 13. As illustrated in FIG. 3B, the central portion 22 may be protruding such that it is received into the cavity 13 when positioned to facilitate sealing.

While this invention is described in association with lumbar vertebrae, it is contemplated that the seal 20 of this invention is suitable for the cervical and thoracic regions of the spine as well. Further, as disclosed supra, the seal 20 is contemplated for use in any location in the body associated with a bony dissection where introduction of BMP into the dissection is indicated.

The seal 20 of the present invention is contemplated to be commercially available in a number of different sizes, shapes and include various attachment means. The seals preferably are packaged in separate sterile packaging and can be arranged on a tray that includes single and multiple protector devices in different sizes and embodiments. The seal 20 may be constructed to be substantially pliable to allow for precise contouring of a seal that is selected from a limited selection of sizes.

The presence of a radiopaque material in the seal 20 permits visualization of the seal 20 by X-ray radiation or the like. In situations where the patient's back pain persists or where subsequent surgery is contemplated, the surgeon is able to determine the position of the seal device of this invention prior to or during a subsequent surgery. The radiopaque substance also allows the surgeon to verify the location where the BMP was introduced as determined from the position of the seal.

It is contemplated that in another preferred embodiment of this invention, the seal 20 is colored. It is contemplated that the selected dye will contrast in color with bone, blood or internal tissues, and thus further facilitate subsequent surgery since the surgeon can rapidly identify the seal 20 during the dissection process. Thus, contrasting colors contemplated for use with this device include shades of blue, green, black, purple, yellow, orange or the like.

In another embodiment of the invention, a fusion cage may also be inserted into the cavity to provide additional structural support prior to new bone formation. The fusion cage may then sealed into a posterior lateral cavity of the annulus 6 in accordance with the invention as described above.

While this invention will be discussed as it relates to spinal surgery, it is contemplated within the scope of this invention that the seal of this invention is suitable as a protective seal for any bony dissection in a vertebrate where BMP has been introduced. Therefore, while a preferred embodiment of this invention relates to the use of the seal to cover a cavity in a vertebrae, the seal device could similarly be used to cover any site where introduction of BMP to foster new bone formation is also indicated. Those with skill in the art of orthopaedics or neurosurgery will be able to generate formed seals, anchorable to bone, that will seal BMP introduction sites in a variety of skeletal tissues.

While particular embodiments of the invention have been described in detail, it will be apparent to those skilled in the art that these embodiments are exemplary rather than limiting, and the true scope of the invention is that defined in the following claims.

What is claimed is:

1. A biocompatible seal device comprising:
a solid sheet, the sheet having a protruding portion configured to fit in an opening in a disk annulus.

2. The device of claim 1 wherein the protruding portion is substantially circular.

3. The device of claim 1 wherein the sheet is substantially pliable.

4. The device of claim 1 wherein the sheet includes a radiopaque material.

5. The device of claim 1 wherein the sheet is prepared in a color that contrasts with the colors of the viscera and skeletal tissue.

6. A method of surgery using a bone morphogenic inducing protein which minimizes unintended post-surgical bone growth, the method comprising:
resecting a posterior lateral herniation in a spinal disk annulus;
forming an opening in the annulus in a posterior lateral location;
introducing bone morphogenic protein (BMP) into the opening;
sealing the opening with a biocompatible seal wherein the seal is positioned to cover the opening so as to inhibit leakage of the BMP into surrounding tissue.

7. The method of claim 6 wherein the seal is substantially pliable.

8. The method of claim 6 wherein sealing the opening includes positioning a protruding portion of the seal into the opening.

9. The method of claim 6 further comprising affixing the seal in place.

10. The method of claim 6 wherein is positioned so that at least a portion of the seal is received into the opening.

11. The method of claim 6 wherein the seal includes a radiopaque material.

12. The method of claim 6 wherein the seal is prepared in a color that contrasts with the colors of the viscera and skeletal tissue.

13. A biocompatible seal device comprising:
a sheet, the sheet having a first surface portion contoured to cover an opening formed in a spinal disk annulus and a second surface portion contoured to align with the exterior surface of the spinal disk
wherein the sheet is composed of a material which inhibits the postoperative migration of surgically introduced bone morphogenic protein into surrounding tissue.

14. The device of claim 13 wherein the sheet is substantially pliable.

15. The device of claim 13 wherein at least a portion of the sheet is received into the opening.

16. The device of claim 13 wherein the sheet includes a radiopaque material.

17. The device of claim 13 wherein the sheet is prepared in a color that contrasts with the colors of the viscera and skeletal tissue.
